(19) United States
(12) Patent Application Publication
(10) Pub. No.: US 2026/0074047 A1
(43) Pub. Date: Mar. 12, 2026

(54) SYSTEM AND METHOD FOR VERTEBRA BONE DENSITY ANALYSIS

(71) Applicant: AGADA Medical LTD., Ramat Hasharon (IL)

(72) Inventors: Isador LIEBERMAN, Plano, TX (US); Samuel SHANNON, Karmei Yosef (IL); Jacov BLANK, Ramat Hasharon (IL); Damon Eugene MAR, Kansas City, KS (US)

(21) Appl. No.: 19/322,272

(22) Filed: Sep. 8, 2025

Related U.S. Application Data
(60) Provisional application No. 63/692,430, filed on Sep. 9, 2024.

(51) Int. Cl. G16H 20/40; G06T 7/00; G06T 7/11; G06T 15/20; G16H 10/60; G16H 30/20; G16H 30/40; G16H 50/20; G16H 50/30; G16H 50/50; G16H 50/70

(52) U.S. Cl. CPC G16H 20/40; G06T 7/0012; G06T 7/11; G06T 15/205; G16H 10/60; G16H 30/20; G16H 30/40; G16H 50/20; G16H 50/30; G16H 50/50; G16H 50/70; G06T 2207/10081; G06T 2207/20081; G06T 2207/20084; G06T 2207/30012

(57) ABSTRACT

Systems, methods, and computer-readable storage media for vertebra bone density analysis, and more specifically to identifying which portions of vertebra have sufficient density to support different types of spinal surgery. A system can segment the pre-operation medical images and generate a 3D pre-operation model having a plurality of volumetric regions based on those pre-operation medical images, wherein each volumetric region has a bone density. The system can then identify different spine surgery options and generate, for each of the plurality of spine surgery options using the 3D pre-operation model, a 3D predicted model. The system can then simulate these predicted models being exposed to various stresses and forces, and predict which of the predicted models results in the best surgery outcome.

### CROSS-REFERENCE

This application claims priority to U.S. provisional patent application No. 63/692,430, filed Sep. 9, 2024, the contents of which are incorporated herein in their entirety.

### BACKGROUND

### 1. Technical Field

The present disclosure relates to vertebra bone density analysis, and more specifically to identifying which portions of vertebra have sufficient density to support different types of spinal surgery.

### 2. Introduction

Just as structural engineers must consider how a structure will support the forces acting on it, a spinal surgeon must analyze the forces that will be acting on the spine and predict the outcome of surgery. To date, that spinal analysis and subsequent prediction regarding surgical outcome have been performed by the surgeons themselves, leading to imprecise and anecdotal analyses and predictions.

### SUMMARY

Additional features and advantages of the disclosure will be set forth in the description that follows, and in part will be understood from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims, or can be learned by the practice of the principles set forth herein.

Disclosed are systems, methods, and non-transitory computer-readable storage media which provide a technical solution to the technical problem described. A method for performing the concepts disclosed herein can include: receiving, at a computer system, a plurality of pre-operation medical images of a patient, the plurality of pre-operation medical images capturing at least one vertebral body; segmenting, via at least one processor of the computer system, the pre-operation medical images, resulting in three-dimensional (3D) pre-operation model of the at least one vertebral body, the 3D pre-operation model comprising a plurality of volumetric regions, wherein each volumetric region in the plurality of volumetric regions has a bone density based at least in part on the plurality of pre-operation medical images; identifying a plurality of spine surgery options; generating, for each of the plurality of spine surgery options using the 3D pre-operation model, at least one 3D predicted model of the at least one vertebral body, resulting in a plurality of 3D predicted models; simulating, via the at least one processor, a plurality of forces being applied to each model in the plurality of 3D predicted models, resulting in a plurality of predicted surgery outcomes; ranking, via the at least one processor, the plurality of spine surgery options based on the plurality of predicted surgery outcomes, resulting in a plurality of risk assessments; and displaying, via a display of the computer system, the plurality of risk assessments.

A system configured to perform the concepts disclosed herein can include: a display; at least one processor; and a non-transitory computer-readable storage medium having instructions stored which, when executed by the at least one processor, cause the at least one processor to perform operations comprising: receiving a plurality of pre-operation medical images of a patient, the plurality of pre-operation medical images capturing at least one vertebral body; segmenting the pre-operation medical images, resulting in three-dimensional (3D) pre-operation model of the at least one vertebral body, the 3D pre-operation model comprising a plurality of volumetric regions, wherein each volumetric region in the plurality of volumetric regions has a bone density based at least in part on the plurality of pre-operation medical images; identifying a plurality of spine surgery options; generating, for each of the plurality of spine surgery options using the 3D pre-operation model, at least one 3D predicted model of the at least one vertebral body, resulting in a plurality of 3D predicted models; simulating a plurality of forces being applied to each model in the plurality of 3D predicted models, resulting in a plurality of predicted surgery outcomes; ranking the plurality of spine surgery options based on the plurality of predicted surgery outcomes, resulting in a plurality of risk assessments; and displaying, via the display, the plurality of risk assessments.

A non-transitory computer-readable storage medium configured as disclosed herein can have instructions stored which, when executed by at least one processor, cause the at least one processor to perform operations which include: receiving a plurality of pre-operation medical images of a patient, the plurality of pre-operation medical images capturing at least one vertebral body; segmenting the pre-operation medical images, resulting in three-dimensional (3D) pre-operation model of the at least one vertebral body, the 3D pre-operation model comprising a plurality of volumetric regions, wherein each volumetric region in the plurality of volumetric regions has a bone density based at least in part on the plurality of pre-operation medical images; identifying a plurality of spine surgery options; generating, for each of the plurality of spine surgery options using the 3D pre-operation model, at least one 3D predicted model of the at least one vertebral body, resulting in a plurality of 3D predicted models; simulating a plurality of forces being applied to each model in the plurality of 3D predicted models, resulting in a plurality of predicted surgery outcomes; ranking the plurality of spine surgery options based on the plurality of predicted surgery outcomes, resulting in a plurality of risk assessments; and displaying, via the display, the plurality of risk assessments.

### BRIEF DESCRIPTION OF THE DRAWINGS

FIG. 1 illustrates an example vertebra with emphasis on the vertebra body;

FIG. 2 illustrates an example vertebra with emphasis on the vertebra pedicles;

FIG. 3 illustrates an example vertebra with emphasis on the upper and lower endplates of the vertebra body;

FIG. 4A illustrates an example vertebra;

FIG. 4B illustrates an example vertebra;

FIG. 4C illustrates an example vertebra;

FIG. 5 illustrates a first exemplary flow chart illustrating how the system generates models and performs risk analysis;

FIG. 6 illustrates a second exemplary flow chart illustrating how the system computes vertebrae and regional statistics and performs risk analysis;

FIG. 7 illustrates an example of a vertebra bone density analysis;

FIG. 8 illustrates an example method embodiment; and

FIG. 9 illustrates an example computer system.

### DETAILED DESCRIPTION

Various embodiments of the disclosure are described in detail below. While specific implementations are described, this is done for illustration purposes only. Other components and configurations may be used without parting from the spirit and scope of the disclosure.

Dual X-Ray Absorptiometry (DEXA) scan is a type of medical imaging test. It uses very low levels of x-rays to measure how dense your bones are by passing a high and low energy x-ray beam (a form of ionizing radiation) through the body, usually in the hip and the spine. This procedure can be used for diagnosing osteoporosis or bone thinning and may be repeated over time to track changes in bone density.

Unfortunately, a DEXA scan is not helpful with respect to spinal surgery. QCT and MRI bone density measures are area based and lack ability to measure volumetric density. For example, performing a DEXA scan on both a bone of uniform density and a hollow box of bone with thick walls can result in identical bone DEXA scores, simply based on the way that DEXA scans are measured. Despite the similar DEXA scan scores, if a surgeon were to put a screw into the hollow box of bone, the screw would not hold the same way as if you put a screw into a uniform piece of bone. In this example, the screw would likely rip out of the hollow box as many of the threads would not be engaged with the bone, whereas a screw placed into a uniform piece of bone would likely remain secure.

By contrast, systems configured as disclosed herein use data provided by Computer Tomography (CT) scans to quantify the density of vertebrae. Preferably, the CT scans provide Hounsfield Units (HU), a quantitative scale for describing radiodensity of body tissues and material. Recent breakthroughs allow translation of Hounsfield Units into bone density directly. For example, the system can estimate that anything above 100 HU is probably better than 0.75 g/cc of hydroxyapatite in the bone.

For example, the CT scans could provide data as illustrated in Table 1:

TABLE 1

| Region | Average | Maximum | Minimum |
|---|---|---|---|
| Left Pedicle | 100 | 130 | 55 |
| Right Pedicle | 50 | 110 | 40 |
| Cephalad ⅓ | 95 | 100 | 80 |
| Centrum | 70 | 125 | 40 |
| Caudal ⅓ | 65 | 85 | 35 |
| Cephalad Endplate | 125 | 130 | 110 |
| Caudal Endplate | 65 | 90 | 45 |

Systems configured as disclosed herein can receive the CT scans, which are quantified in planes. The CT scan planes (generally) are in the axial plane (also known as the transverse plane, horizontal plane, or transaxial plane), which is an anatomical plane that divides the body into superior (portions above) and inferior (portions below) sections. Using a series of neighboring planes of CT scans, the system can: (1) generate a three-dimensional (3D) volumetric model of a patient's spine, where the 3D volumetric model identifies the regional densities of specific portions of a vertebra or vertebrae; and (2) generate a virtual patient model, where the 3D volumetric model of the spine, a 3D patient-specific spine segmentation, as well as ligaments, discs, muscles, and/or other portions of the patient, are combined together to create a virtual “twin” of the patient. The regional densities correspond to volumetric regions, and represent regions having 3D volumes (i.e., a height, width, and depth). These volumetric regions can have other qualities as well-specifically density.

Generation of the 3D volumetric model of the patient's spine can occur via a segmentation algorithm, where the system analyzes each image within the CT scan (a typical CT scan includes many individual images, and can be part of a medical study where a number of images of an individual (i.e., a patient) are taken) to identify each individual vertebra within the CT scan. Preferably, systems configured as disclosed herein rely on Artificial Intelligence (AI) segmentation in the form of neural networks, convolutional neural networks (CNN), graph convolutional networks (GCN), and/or point clouds (PC) to analyze bone structures within the CT scan. The result of this segmentation is the vertebrae of the patient being identified, with each vertebra having a determined volume and various dimensions. Within each vertebra, the volumetric bone density for specific regions within the vertebra is calculated based on the HU values provided by the CT scan (as discussed above). The volumes for the specific regions can vary depending on scale and user preference, such that the number of regions within any given vertebra can vary as needed. Combined together, the vertebrae form the 3D volumetric model of the patient's spine. Preferably, the 3D volumetric model also contains anthropometric data about the patient, such as (but not limited to) age, height, weight, head circumference, body mass index (BMI), and/or skinfold thickness. This data is separate from segmented 3D volumetric information (e.g., from the segmented 3D volumetric model), and is instead accessed from the patient's electronic medical record.

In generating the virtual patient model, the system uses the 3D volumetric model of the spine, as well as estimated locations of soft tissues (estimated according to a generic model), to create a virtual “twin” of the patient. This virtual patient model can then be used to simulate stresses being applied to the patient, such that the system can determine how the patient's body, including the vertebrae, would respond to different forces, torques, stresses, strain, etc. Non-limiting potential results from such stress simulations can include a likelihood of vertebrae fracture, a likelihood of subsidence (a gradual caving in of the vertebra), and/or a screw pullout risk. Such simulations can be, for example, based on movement of an individual while undergoing different movements or otherwise being exposed to stresses, and can sequentially process through each stage of the movement. If, for example, the system is simulating a patient walking after undergoing a particular medical procedure, the system can sequentially simulate the forces on the patient's body/vertebrae for distinct points during the walking process (e.g., how much pressure/rotational force/torque/etc. is on the vertebrae when the left foot is striking, when the left foot rises, when the left foot pushes off, etc.?). From a computational standpoint the individual steps of the simulation process will always be discrete, though from a practical standpoint at a certain level the individual steps of the simulation process become virtually continuous.

With the 3D volumetric model of the patient's spine and the virtual patient model generated, the system can perform a risk analysis of one or more different types of surgery using different locations for anchor points, as needed by the different surgeries. For example, the system can, for a surgery requiring a screw to be inserted into a vertebra, analyze inserting the screw into different parts of the vertebra based on the bone density within a specific region. If, for example, a surgery required insertion of a screw into a pedicle of the vertebra, but one pedicle had a relatively low bone density compared to the other, the system analysis could recommend the screw be inserted into the pedicle with the higher bone density. Alternatively, the system can provide the surgeon with information about both pedicle, along with associated risks, and the surgeon can make a determination regarding the placement of the respective screw, or the need to augment the screw purchase with cement. Likewise, the risk analysis can include different angles of surgery or implantation, different locations of the surgery (e.g., testing which spot on a pedicle of the vertebra would be best by testing simulation/providing a risk analysis of different locations on the pedicle located a predefined distance from one another (e.g., 3 mm)), different types of surgery, different types of hardware, etc.

The 3D volumetric model of the spine and the virtual patient model can be pre-operation, or “pre-op”, models. The system can then generate, based on the pre-op models (and the bone densities within those models), a likely post-operation, or “post-op”, model or predicted outcome for (1) each different placement of screws, rods, and/or other medical devices and (2) each type of medical procedure available. In some cases, the doctor or surgeon can select which medical procedures are evaluated by the system before the post-op outcome(s) is generated, whereas in other cases, the system has the medical procedures pre-defined.

To make the post-op predictions, the system can use Artificial Intelligence (AI), such as (but not limited to) a neural network, computer vision, or other software capable of making a weighted, multi-layered correlation that can change/be updated over time. Here, the AI is trained using previous procedures and their respective outcomes. Non-limiting examples of training data can include: patient diagnosis, the pre-op/post-op 3D volumetric models for a patient, the pre-op/post-op virtual patient models for the patient, the type of procedure performed, the pre-op/post-op bone density information associated with both the pre-op/post-op 3D volumetric model and the virtual patient model, pre-op/post-op bone density where a medical device was inserted, outcomes (e.g., a screw held; a screw has movement; the patient remains in pain; the patient can no longer walk; etc.) and/or any other data about the patient (e.g., age, weight, sex, objective/functional gait analysis, measured walking tolerance, subjective data including patient reported outcome measures (e.g., subjective pain level (e.g., “My pain level is [a user provided number]”) or walking tolerance (e.g., “Pain becomes unbearable after five minutes of walking”)), etc.). Using that training data, the AI can predict, for a current patient, how each possible procedure is likely to affect the patient's diagnosis and/or surgery outcome. Based on those results, the system can rank the procedures and display the likely outcomes for each procedure for review by the doctor. The ranking can, in some configurations, be a list (e.g., A is the best surgery option, B is the second best option . . . , G is the worst option). In other configurations, the ranking can be a comparison between the likely outcomes (e.g., A is the most likely to have a positive outcome, B is the second most likely, etc.). In still other configurations, such as where the surgery options can be ranked on multiple factors (e.g., cost, likely outcome, surgical risk, risk of subsequent problems, etc.), the system can allow a user to define the weights associated with each of factors, then rank the surgery options based on the sum of those weighted factors.

In some cases, the spine surgery option is selected, at least in part, based on which of the plurality of spine surgery options is most likely to reduce the likelihood of failure due to bone collapse or screw pullout. For example, a given patient may have different bone densities within the vertebral body and between vertebral bodies predisposing to failure. There may be optimal screw positioning to mitigate this occurrence, or the surgeon may elect to augment the screw purchase with bone cement. These variables, along with the additional factors, such as, e.g., the patient's age, the level of intrusion/difficulty of the surgery, the patient's ability to recover from the surgery, etc., may also be used by the system in ranking and determining the available procedures.

After a procedure has taken place, additional medical images can be taken, and a post-op 3D volumetric model of the spine can be generated. This new, post-op 3D model can be compared against the predicted post-op 3D volumetric model for that procedure, which was previously generated, as well as against the pre-op 3D volumetric model. This comparison data, as well as data about the procedure (e.g., type of procedure, data about the patient, bone density, etc.), can then be used as updated training data for the AI system. The AI system can be updated after each procedure, or can be updated after a predetermined amount of time or a predetermined number of procedures have occurred.

The training of the neural network or AI system can occur by any means known to those of skill in the art. Preferably, the neural network/AI system is trained on historical data associated with previous surgeries, models, and known outcomes (e.g., Was the previous surgery associated with the provided data successful, or create the desired effects? Did the previous surgery result in a satisfied patient? Did the previous surgery require additional, unplanned surgeries?).

In some configurations, the system disclosed can, upon identifying the best medical procedure for a given patient, initiate the medical procedure. In some configurations this can involve directing a doctor or surgeon to perform the selected (best) medical procedure. In other cases, this can involve directing a robot, surgical tools, or other mechanized instruments to begin the medical procedure (e.g., without human direction, though possibly with human oversight).

In this manner, the system disclosed herein (1) improves accuracy in determining where screws, rods, and/or other medical devices should be placed for a given patient; (2) identifies the best medical procedure (predicted outcome and/or patients' symptom relief, along with optimizing the biomechanics of vertebral loading) available for a given patient; and (3) improves, over time, the post-op predictions.

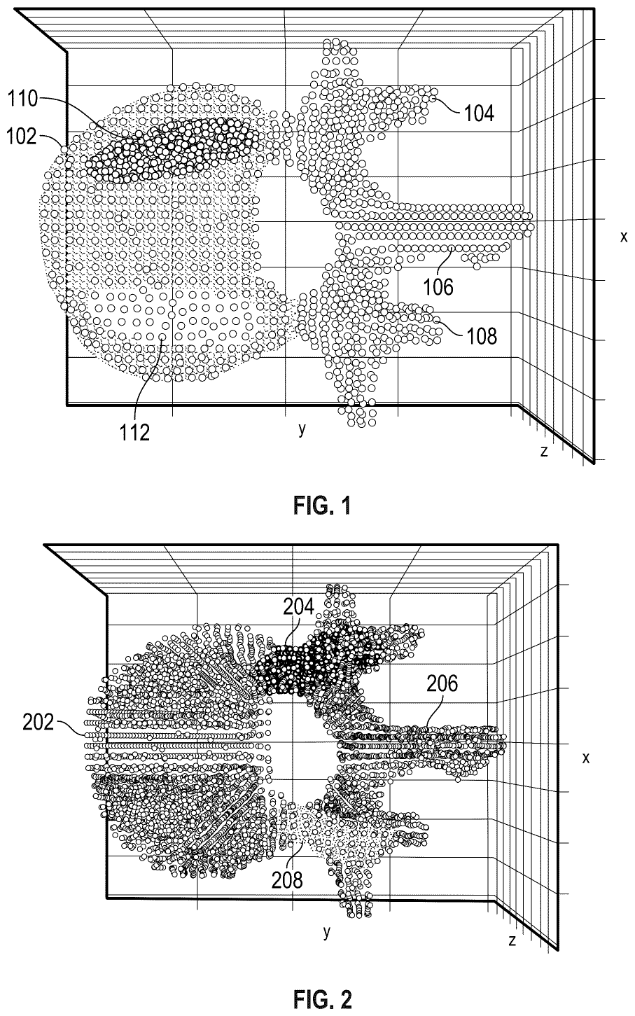

FIG. 1 illustrates an example vertebra with emphasis on the vertebra body 102. In this example, bone density is illustrated within different regions of the vertebra by the number of small circles on the vertebra. Small circles are used purely for illustration purposes, with the understanding that other shapes, colors, or designations for showing viable portions of bone may vary in specific configurations. In practice, shading, different shapes, numbers, or any other mechanisms known to a person of ordinary skill in the art may be used to display or otherwise illustrate bone density. In this example, the system analyzes a vertebra and determines that one portion 110 of the vertebral body 102 has higher bone density (illustrated by a higher number/density of small circles) compared to a second portion 112 of the vertebral body 102 (illustrated by fewer small circles, representing a lower density), while the bone density of the transverse processes 104, 108 and the spinous process 106 are all of medium density (i.e., denser than the second portion 112, but not as dense as the first portion 110). In this example, the first section 110 would be preferable (because of the higher density) for implanting a device over the other portions of the vertebra. Reasons why the bone density may not be ideal, or may be less ideal than another location, may include the bone density being too low or too high. Normally one should expect some variability in the density within the vertebral body 102 which is associated with vertical trabecular columns serving as supports between the end plates. In addition, the density analysis may be used by the system to make a recommendation for use of cement or other mechanisms for improving the likelihood that an implant will hold.

FIG. 2 illustrates an example vertebra with emphasis on the vertebra pedicles. In this example, a given surgical procedure needs to attach a medical device (such as a screw or rod) to a pedicle, such that the bone density of the vertebra body 202 and the spinous process 206 are not taken into consideration. In this example, the bone density for the “top” pedicle 204 is not ideal for this given surgery, whereas the bone density for the “bottom” pedicle 208 is ideal, or at least more ideal (see, e.g., the close and non-uniform spacing of the circles in the illustrated “top” pedicle 204, as compared to the relatively uniform spacing of the circles in the illustrated “bottom” pedicle 208).

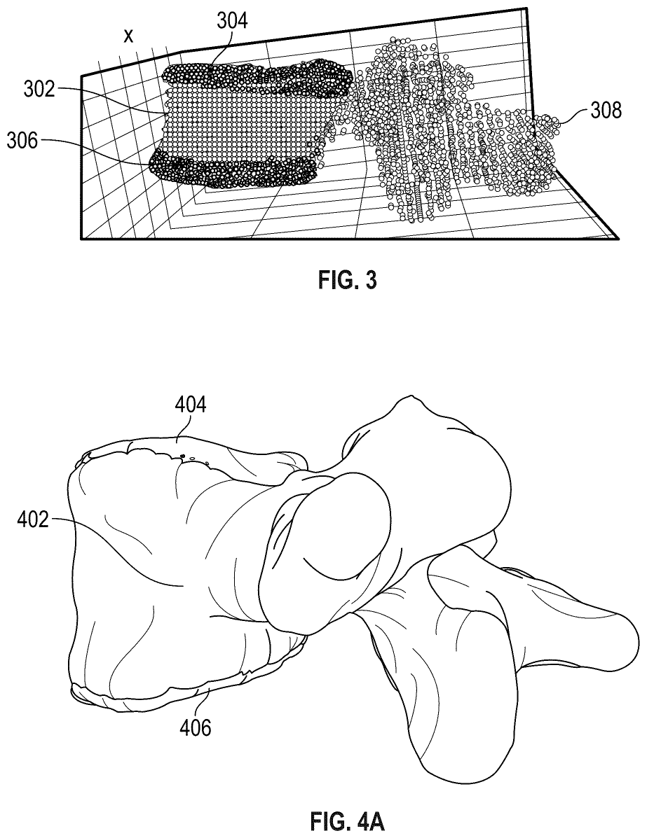

FIG. 3 illustrates an example vertebra with emphasis on the upper and lower endplates of the vertebra body. In this example, a given surgical procedure needs to attach a medical device to the endplates 304, 306 of the vertebra body 302, with the remainder of the vertebra 308 not being taken into consideration. In this example, both endplates 304, 306 are identified as weak, or otherwise incapable of supporting the medical device required for the given surgical procedure, due to the bone density being illustrated. In practice, the endplates 304, 306 are identified as weak not solely based on the density within endplates 304, 306, but also based on the trabecular support from 302 which supports endplates 304, 306 (i.e., the illustration shows a simplified version of the actual analysis taking place to determine bone density, where the full analysis takes into account the entire vertebra, and how segments of that vertebra relate to one another). In this example, the system would likely recommend that this surgery not be performed, at least in the manner initially proposed.

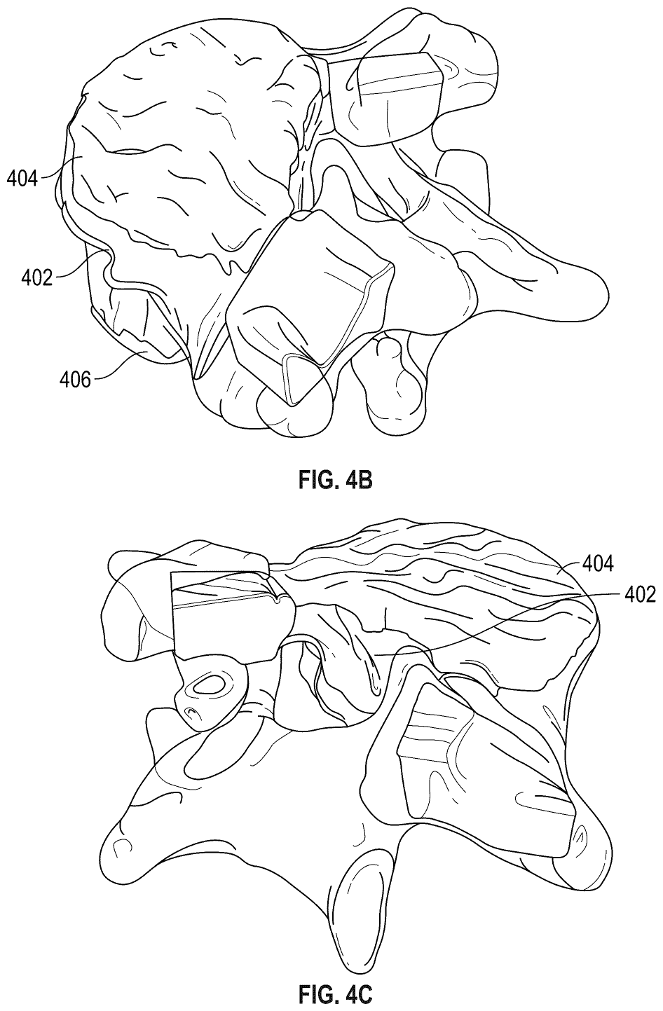

FIGS. 4A, 4B, and 4C illustrate an example vertebra. These example vertebra again illustrates the endplates 404, 406 on either side of a vertebra body 402.

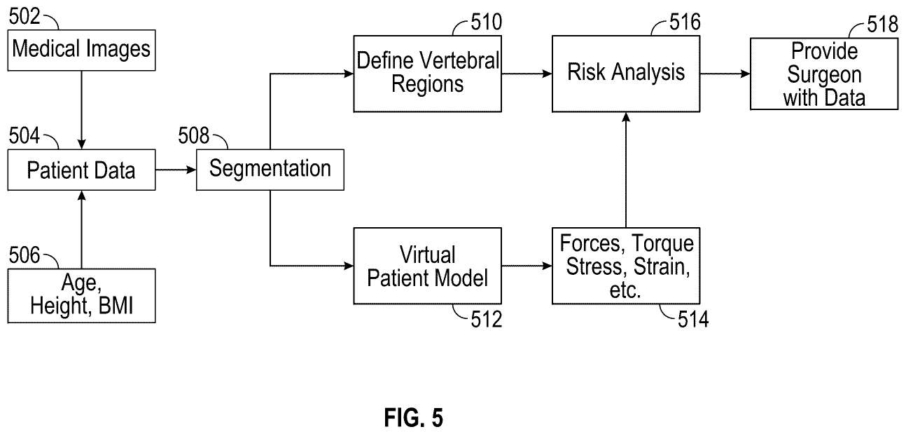

FIG. 5 illustrates a first exemplary flow chart illustrating how the system generates models and performs risk analysis. In this example, the system receives patient data 504 made up of medical images 502 (such as a CT scan consisting of multiple images, MRI images and/or X-ray images), as well as anthropometric data about the patient, such as (but not limited to) age, height, weight, head circumference, body mass index (BMI), and/or skinfold thickness. The medical images 502 can capture at least the vertebral body in question, with the vertebral body including at least the cephalad (toward the head) and caudad (toward the feet) vertebra around the vertebra in question. Alternatively, the medical images 502 can capture the entirety of the spine, or all vertebrae in a spinal region. In some configurations, in addition to the at least one vertebral body, the images can capture one or more functional spinal units. A functional spinal unit can further include the intervening tissues between vertebra and any discs.

The system receives the patient data 504 and performs segmentation 508 on the medical images 502, allowing the system to define vertebral volumetric regions 510. While the defining of vertebral volumetric regions 510 can include the identification of vertebrae within the medical images 502, the defining of the vertebral volumetric regions 510 can further include identifying the volume of a given vertebra and identifying specific sub-volumes, also known as volumetric regions, within the vertebra. If there are vertebrae, the defining of the vertebral volumetric regions 510 can be done for all of the vertebrae or a specified subsection thereof. In defining the vertebral volumetric regions 510, the system can generate a three-dimensional (3D) model of the patient's spine, a portion of the patient's spine (e.g., a functional spinal unit), or any combination thereof. In defining the vertebral volumetric regions 510, the system can also compute vertebrae and region HU statistics (or values), as discussed above, and/or compute cortical bone thickness.

The system can also generate, based on the segmentation 508 and the patient data 504, a virtual patient model 512. In some configurations, the defining of the virtual patient model 512 can be done using the defined vertebral regions 510 and/or a generated 3D model. The virtual patient model 512 can include a spine, as well as ligaments, tendons, discs, muscles, and/or other parts of a patient's anatomy. The system can then expose that virtual patient model 512 to forces, torque, stress, strain, etc. 514 to determine how the virtual patient model will respond in various scenarios. Preferably, the forces, torque, stress, strain, etc. 514 are used to predict how a given patient will respond to a given surgery, and more specifically, how the patient may respond to specific positioning of medical devices (screws, rods, etc.) being implanted on/in the spine. In this manner, the system can simulate the outcomes of different types of surgeries, and/or different locations for placing screws, rods, etc., on the vertebrae, and record the predicted outcomes of those surgeries.

The system then executes a risk analysis 516 based on the defined vertebral regions 510 and/or 3D model, as well as the results of the forces, torque, stress, strain, etc. 514 test or simulation through which the virtual patient model 512 was exposed. The risk analysis 516 can, for example, rank the outcomes of the various surgeries based on desired results, and provide the surgeon with the data 518 based on this risk analysis 516. The surgeon can then consider the predicted outcomes when determining which surgery to perform, and how to perform it.

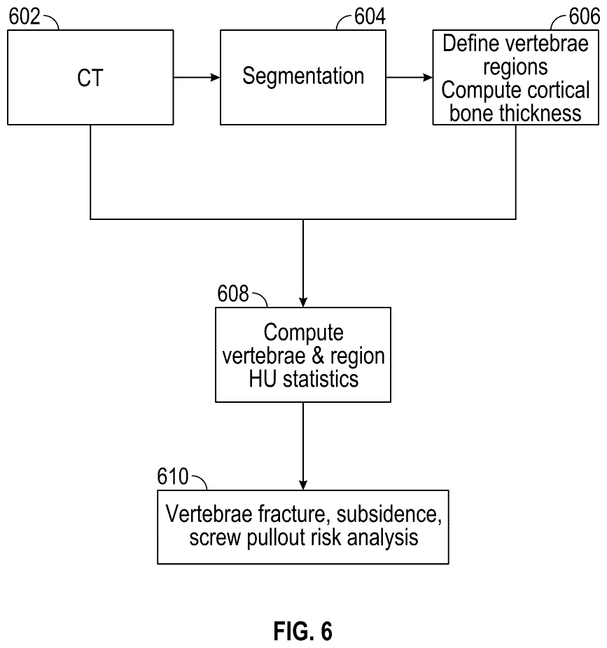

FIG. 6 illustrates a second exemplary flow chart illustrating how the system computes vertebrae and regional statistics and performs risk analysis. In this example, the system receives CT scan images 602 of a patient, then performs segmentation 604 on the CT scan images 602, resulting in defined vertebrae volumetric regions 606, where the defined vertebrae volumetric regions 606 have computed cortical bone thickness. The system then computes, using the CT scan images 602 and the defined vertebral volumetric regions 606, vertebrae and regional HU statistics 608, which can be converted into bone density for the defined vertebrae volumetric regions. For example, the vertebra can be divided into different volumetric regions and HU can be calculated using statistical analysis in each volumetric region and in the whole vertebra. According to the HU values calculated, the bone density of each vertebra and each volumetric region can be estimated. According to one embodiment, the vertebra shell thickness and the regional shell thickness can also be calculated. The system can then perform, using the bone density, a vertebrae fracture, subsidence, and/or screw pullout risk analysis 610. In other words, the system can predict possible negative outcomes associated with a possible surgery to assist a surgeon with spinal surgical planning, including, for example, which vertebra and/or volumetric region to cement, use and/or type of a cage, implant size, footprint, type of implant, etc.

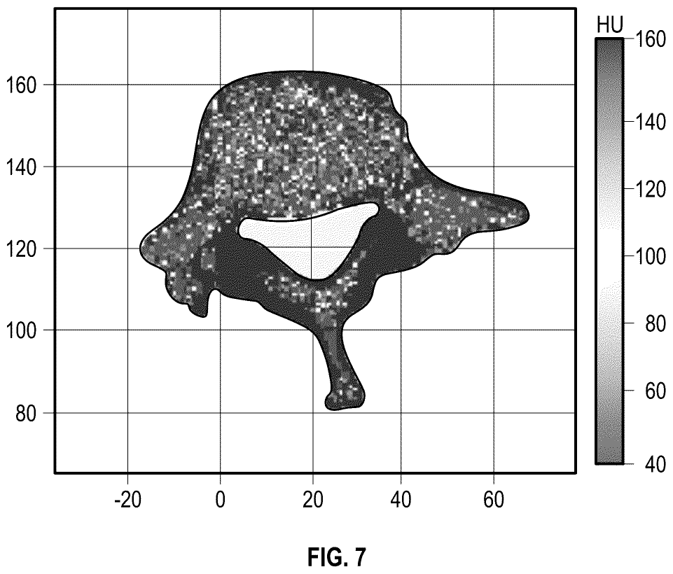

FIG. 7 illustrates an example of a vertebra bone density analysis. As illustrated, some portions of the vertebra have a higher density level than others. The system can use these densities in evaluating surgery options to identify which procedures will have a highest likelihood of success. In practice, the system can identify preferred locations for medical device placement based on a combination of location and HU values—not just based on density. The location of favorable pixels provided to the surgeon is a combination of location, distribution, and consistency.

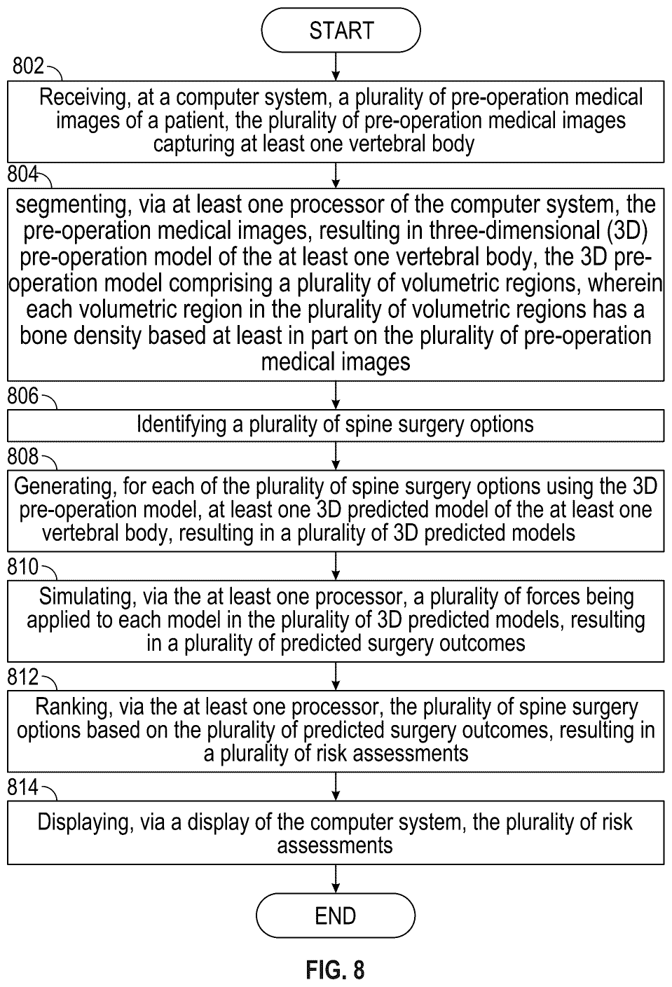

FIG. 8 illustrates an example method embodiment of a computer system, configured as disclosed herein, being used to provide risk assessments of a plurality of spine surgery options. As illustrated, the computer system can receive a plurality of pre-operation medical images of a patient, the plurality of pre-operation medical images capturing at least one vertebral body (802), and segment the pre-operation medical images, resulting in a three-dimensional (3D) pre-operation model of the at least one vertebral body, the 3D pre-operation model comprising a plurality of volumetric regions (i.e., the region having a 3D volume, with height, length, and width), wherein each volumetric region in the plurality of volumetric regions has a bone density based at least in part on the plurality of pre-operation medical images (804). The system can then identify (automatically based on the needs of a patient, or based on inputs from a doctor) a plurality of spine surgery options (806), and generate, for each of the plurality of spine surgery options using the 3D pre-operation model, at least one 3D predicted model of the at least one vertebral body, resulting in a plurality of 3D predicted models (808). The system can then simulate a plurality of forces being applied to each model in the plurality of 3D predicted models, resulting in a plurality of predicted surgery outcomes (810) and rank the plurality of spine surgery options based on the plurality of predicted surgery outcomes, resulting in a plurality of risk assessments (812). The system can then display the plurality of risk assessments (814).

In some configurations, the generating, for each of the plurality of spine surgery options, of the at least one 3D predicted model can further include: identifying a plurality of possible locations within the 3D pre-operation model where one or more virtual medical devices can be virtually inserted; and generating, for each of the plurality of possible locations, a distinct model in the plurality of 3D predicted models, such that for each spine surgery option in the plurality of spine surgery options, multiple 3D predicted models are generated, each of the multiple 3D predicted models having the virtual medical device(s) inserted at distinct locations. In such configurations, the distinct locations can have distinct bone density.

In some configurations, the plurality of 3D predicted models can further include anthropometric data about the patient.

In some configurations, the illustrated method can further include: generating, via the at least one processor using the plurality of pre-operation medical images and the 3D pre-operation model, a virtual patient model, the virtual patient model comprising virtual representations of the at least one functional spinal unit and at least one of a ligament, a disc, and a muscle; and generating, for each of the plurality of spine surgery options using the a plurality of 3D predicted models and the virtual patient model, at least one predicted virtual model of the patient, resulting in a plurality of predicted virtual models, wherein the simulating of the plurality of forces is further applied to the plurality of predicted virtual models.

In some configurations, the simulating is performed by the at least one processor executing a neural network, wherein the neural network is trained on previous spine surgery predictions and associated outcomes. In such configurations, the illustrated method can further include: receiving, at the computer system after execution of a selected spine surgery option, a plurality of post-operation medical images, the plurality of post-operation medical images capturing the at least one functional spinal unit; receiving, at the computer system after execution of the selected spine surgery option, a patient outcome of the selected spine surgery option; segmenting, via the at least one processor, the post-operation medical images, resulting in a 3D post-operation model of the at least one functional spinal unit; comparing, via the at least one processor, the 3D post-operation model to a specific 3D predicted model within the plurality of 3D predicted models, the specific 3D predicted model being for the selected spine surgery option, resulting in a model comparison; comparing, via the at least one processor, the patient outcome to a predicted surgery outcome within the plurality of predicted surgery outcomes, resulting in an outcome comparison; and updating the neural network based on the model comparison and the outcome comparison.

In some configurations, the simulating identifies at least one of: (A) a likelihood of vertebrae fracture, (B) a likelihood of subsidence, or (C) a screw pullout risk analysis.

In some configurations, the bone density for each 3D region are received in Hounsfield units (HU), then converted by the at least one processor to grams/cubic centimeter (g/cc) of hydroxyapatite.

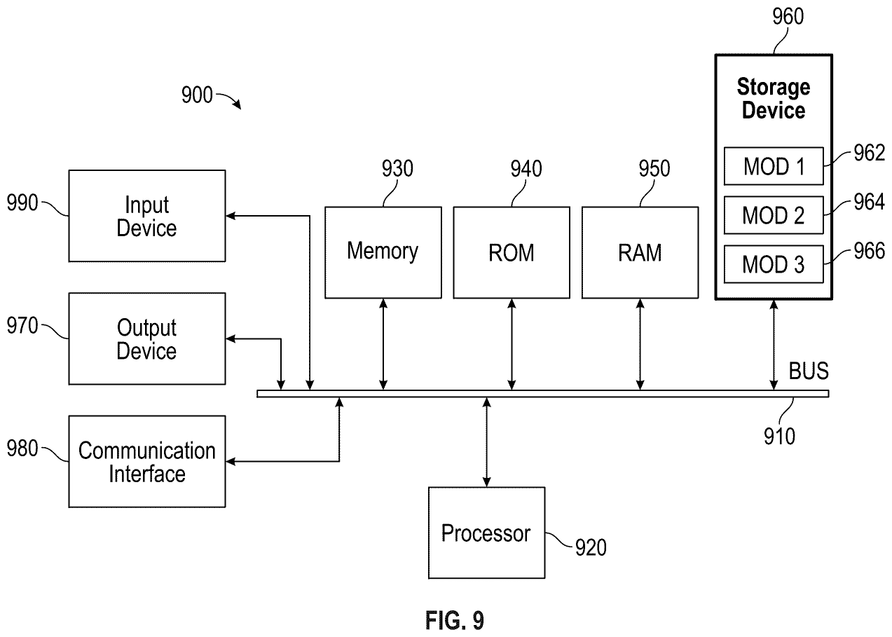

With reference to FIG. 9, an exemplary system includes a computing device 900 (such as a general-purpose computing device), including a processing unit (CPU or processor) 920 and a system bus 910 that couples various system components including the system memory 930 such as read-only memory (ROM) 940 and random access memory (RAM) 950 to the processor 920. The computing device 900 can include a cache of high-speed memory connected directly with, in close proximity to, or integrated as part of the processor 920. The computing device 900 copies data from the system memory 930 and/or the storage device 960 to the cache for quick access by the processor 920. In this way, the cache provides a performance boost that avoids processor 920 delays while waiting for data. These and other modules can control or be configured to control the processor 920 to perform various actions. Other system memory 930 may be available for use as well. The system memory 930 can include multiple different types of memory with different performance characteristics. It can be appreciated that the disclosure may operate on a computing device 900 with more than one processor 920 or on a group or cluster of computing devices networked together to provide greater processing capability. The processor 920 can include any general-purpose processor and a hardware module or software module, such as module 1 962, module 2 964, and module 3 966 stored in storage device 960, configured to control the processor 920 as well as a special-purpose processor where software instructions are incorporated into the actual processor design. The processor 920 may essentially be a completely self-contained computing system, containing multiple cores or processors, a bus, memory controller, cache, etc. A multi-core processor may be symmetric or asymmetric.

The system bus 910 may be any of several types of bus structures including a memory bus or memory controller, a peripheral bus, and a local bus using any of a variety of bus architectures. A basic input/output (BIOS) stored in memory ROM 940 or the like, may provide the basic routine that helps to transfer information between elements within the computing device 900, such as during start-up. The computing device 900 further includes storage devices 960 such as a hard disk drive, a magnetic disk drive, an optical disk drive, tape drive or the like. The storage device 960 can include software modules 962, 964, 966 for controlling the processor 920. Other hardware or software modules are contemplated. The storage device 960 is connected to the system bus 910 by a drive interface. The drives and the associated computer-readable storage media provide nonvolatile storage of computer-readable instructions, data structures, program modules and other data for the computing device 900. In one aspect, a hardware module that performs a particular function includes the software component stored in a tangible computer-readable storage medium in connection with the necessary hardware components, such as the processor 920, system bus 910, output device 970 (such as a display or speaker), and so forth, to carry out the function. In another aspect, the system can use a processor and computer-readable storage medium to store instructions which, when executed by a processor (e.g., one or more processors), cause the processor to perform a method or other specific actions. The basic components and appropriate variations are contemplated depending on the type of device, such as whether the computing device 900 is a small, handheld computing device, a desktop computer, or a computer server.

Although the exemplary embodiment described herein employs the storage device 960 (such as a hard disk), other types of computer-readable media which can store data that are accessible by a computer, such as magnetic cassettes, flash memory cards, digital versatile disks, cartridges, random access memories (RAMs) 950, and read-only memory (ROM) 940, may also be used in the exemplary operating environment. Tangible computer-readable storage media, computer-readable storage devices, or computer-readable memory devices, expressly exclude media such as transitory waves, energy, carrier signals, electromagnetic waves, and signals per sc.

To enable user interaction with the computing device 900, an input device 990 represents any number of input mechanisms, such as a microphone for speech, a touch-sensitive screen for gesture or graphical input, keyboard, mouse, motion input, speech and so forth. An output device 970 can also be one or more of a number of output mechanisms known to those of skill in the art. In some instances, multimodal systems enable a user to provide multiple types of input to communicate with the computing device 900. The communications interface 980 generally governs and manages the user input and system output. There is no restriction on operating on any particular hardware arrangement and therefore the basic features here may easily be substituted for improved hardware or firmware arrangements as they are developed.

The technology discussed herein refers to computer-based systems and actions taken by, and information sent to and from, computer-based systems. One of ordinary skill in the art will recognize that the inherent flexibility of computer-based systems allows for a great variety of possible configurations, combinations, and divisions of tasks and functionality between and among components. For instance, processes discussed herein can be implemented using a single computing device or multiple computing devices working in combination. Databases, memory, instructions, and applications can be implemented on a single system or distributed across multiple systems. Distributed components can operate sequentially or in parallel.

Neural networks, foundational to modern artificial intelligence, are computational systems designed to process data and generate predictions or classifications by emulating aspects of human brain function. A neural network is a framework of machine learning algorithms that work together to classify inputs based on a previous training process. They power applications like image recognition, natural language processing, and predictive analytics. At their core, neural networks consist of interconnected layers of mathematical units called neurons, organized into an input layer, one or more hidden layers, and an output layer. The input layer receives raw or preprocessed data, such as pixel values or text embeddings, represented as numerical vectors. Hidden layers transform this data into increasingly abstract representations through complex computations, while the output layer produces the result, such as a class probability or a numerical prediction. Each neuron connects to those in the next layer via weighted connections, where weights are numerical values that amplify or diminish the influence of one neuron's output on another's input. Additionally, biases-adjustable offsets-enhance the model's flexibility in fitting data.

The operation of a neural network begins with a forward pass, where data flows from the input layer through the hidden layers to the output. Each neuron computes a weighted sum of its inputs, adds its bias, and applies a nonlinear activation function, such as a sigmoid, rectified linear unit (ReLU), or hyperbolic tangent (tanh), to produce an output. This process repeats across layers, with each layer extracting more complex features, such as edges in images or semantic patterns in text. The final layer's output depends on the task: classification tasks yield probabilities (e.g., “90%”), while regression tasks produce continuous values (e.g., a predicted temperature). Crucially, the forward pass does not alter the model's stored parameters-weights and biases-which represent the network's learned knowledge. These parameters are stored in digital memory, typically as 32-bit or 16-bit floating-point arrays. Weights form matrices, with rows and columns corresponding to neurons in adjacent layers, while biases are stored as one-dimensional arrays. Meta-information, such as layer counts and activation function types, is also stored to define the network's structure.

Training a neural network involves adjusting its parameters to minimize prediction errors. During training, a forward pass generates predictions, which are compared to correct outputs using a loss function, such as mean squared error or cross-entropy, to quantify errors. Backpropagation then computes gradients, indicating how much each parameter contributed to the error, by applying the chain rule to propagate errors backward from the output to the input layer. Optimization algorithms, like stochastic gradient descent, adjust weights and biases in directions that reduce the loss. This process iterates over multiple epochs, with parameters gradually converging to values that improve accuracy. Memory usage during training is dynamic: weights and biases are updated incrementally for each data batch, and intermediate results, like neuron activations and gradients, are temporarily stored in buffers to facilitate backpropagation. To ensure progress is saved, parameters are periodically checkpointed to persistent storage, allowing training to resume later. Efficiency techniques, such as reducing parameter precision to 16-bit formats, further optimize memory and computation.

Once trained, the network enters inference mode, where parameters are fixed, and only forward passes are executed to generate predictions. This mode minimizes memory writes, making it ideal for deployment on resource-constrained devices like mobile phones. Neural networks can reduce memory usage, use unique parameter update mechanisms to enhance training efficiency, use hybrid memory systems combining volatile and non-volatile storage, and/or perform dynamic precision adjustments during training or inference.

Use of language such as “at least one of X, Y, and Z,” “at least one of X, Y, or Z,” “at least one or more of X, Y, and Z,” “at least one or more of X, Y, or Z,” “at least one or more of X, Y, and/or Z,” or “at least one of X, Y, and/or Z,” are intended to be inclusive of both a single item (e.g., just X, or just Y, or just Z) and multiple items (e.g., {X and Y}, {X and Z}, {Y and Z}, or {X, Y, and Z}). The phrase “at least one of” and similar phrases are not intended to convey a requirement that each possible item must be present, although each possible item may be present.

The various embodiments described above are provided by way of illustration only and should not be construed to limit the scope of the disclosure. Various modifications and changes may be made to the principles described herein without following the example embodiments and applications illustrated and described herein, and without departing from the spirit and scope of the disclosure. For example, unless otherwise explicitly indicated, the steps of a process or method may be performed in an order other than the example embodiments discussed above. Likewise, unless otherwise indicated, various components may be omitted, substituted, or arranged in a configuration other than the example embodiments discussed above.

Spinal surgical planning according to vertebrae reginal bone density, as disclosed herein, can be accomplished by dividing the vertebra into different regions and calculating a statistical analysis of Hounsfield Units (HU) in each region and in the whole vertebra. According to the HU, the system can estimate the bone density of each vertebra and each region. The vertebra shell thickness and the regional shell thickness is calculated. According to these parameters, together and alone, the spine surgery is planned. This planning may include: vertebra and regions to cement, size footprint and type of cage and implants, and/or type and size of the vertebra screw.

Further aspects of the present disclosure are provided by the subject matter of the following clauses.

A method comprising: receiving, at a computer system, a plurality of pre-operation medical images of a patient, the plurality of pre-operation medical images capturing at least one vertebral body; segmenting, via at least one processor of the computer system, the pre-operation medical images, resulting in three-dimensional (3D) pre-operation model of the at least one vertebral body, the 3D pre-operation model comprising a plurality of volumetric regions, wherein each volumetric region in the plurality of volumetric regions has a bone density based at least in part on the plurality of pre-operation medical images; identifying a plurality of spine surgery options; generating, for each of the plurality of spine surgery options using the 3D pre-operation model, at least one 3D predicted model of the at least one vertebral body, resulting in a plurality of 3D predicted models; simulating, via the at least one processor, a plurality of forces being applied to each model in the plurality of 3D predicted models, resulting in a plurality of predicted surgery outcomes; ranking, via the at least one processor, the plurality of spine surgery options based on the plurality of predicted surgery outcomes, resulting in a plurality of risk assessments; and displaying, via a display of the computer system, the plurality of risk assessments.

The method of any preceding clause, further comprising: training a neural network on previous spine surgery predictions and associated outcomes, wherein the simulating is performed by the at least one processor executing the neural network.

The method of any preceding clause, further comprising: selecting, based on the plurality of risk assessments, a selected spine surgery from the plurality of spine surgery options, the selected spine surgery having a best ranking within the plurality of risk assessments; and performing the selected spine surgery. The best ranking can, be determined based on factors such as, but not limited to, least complications, least likely to result in adverse outcomes, most likely to result in a positive outcome, smallest duration, and/or least complicated.

The method of any preceding clause, wherein performing the selected spine surgery can include causing a robotic machine to perform at least one step of the selected spine surgery.

The method of any preceding clause, wherein the generating, for each of the plurality of spine surgery options, of the at least one 3D predicted model further comprises: identifying a plurality of possible locations within the 3D pre-operation model where one or more virtual medical devices can be virtually inserted; and generating, for each of the plurality of possible locations, a distinct model in the plurality of 3D predicted models, such that for each spine surgery option in the plurality of spine surgery options, multiple 3D predicted models are generated, each of the multiple 3D predicted models having the one or more virtual medical devices inserted at distinct locations.

The method of any preceding clause, wherein the distinct locations have distinct bone density.

The method of any preceding clause, wherein the plurality of 3D predicted models further comprise anthropometric data about the patient.

The method of any preceding clause, further comprising: generating, via the at least one processor using the plurality of pre-operation medical images and the 3D pre-operation model, a virtual patient model, the virtual patient model comprising virtual representations of the at least one vertebral body and at least one of a ligament, a disc, and a muscle; and generating, for each of the plurality of spine surgery options using the plurality of 3D predicted models and the virtual patient model, at least one predicted virtual model of the patient, resulting in a plurality of predicted virtual models, wherein the simulating of the plurality of forces is further applied to the plurality of predicted virtual models.

The method of any preceding clause, wherein the simulating is performed by the at least one processor executing a neural network, wherein the neural network is trained on previous spine surgery predictions and associated outcomes.

The method of any preceding clause, further comprising: receiving, at the computer system after execution of a selected spine surgery option, a plurality of post-operation medical images, the plurality of post-operation medical images capturing the at least one vertebral body; receiving, at the computer system after execution of the selected spine surgery option, a patient outcome of the selected spine surgery option; segmenting, via the at least one processor, the post-operation medical images, resulting in a 3D post-operation model of the at least one vertebral body; comparing, via the at least one processor, the 3D post-operation model to a specific 3D predicted model within the plurality of 3D predicted models, the specific 3D predicted model being for the selected spine surgery option, resulting in a model comparison; comparing, via the at least one processor, the patient outcome to a predicted surgery outcome within the plurality of predicted surgery outcomes, resulting in an outcome comparison; and updating the neural network based on the model comparison and the outcome comparison.

The method of any preceding clause, wherein the simulating identifies at least one of: (A) a likelihood of vertebrae fracture, (B) a likelihood of subsidence, or (C) a screw pullout risk analysis.

The method of any preceding clause, wherein the bone density for each 3D region is received in Hounsfield units (HU), then converted by the at least one processor to grams/cubic centimeter (g/cc) of hydroxyapatite.

The method of any preceding clause, wherein: the pre-operation medical images further capture at least one functional spinal unit; and the plurality of 3D predicted models are further based on the at least one functional spinal unit.

A system comprising: at least one processor; and a non-transitory computer-readable storage medium having instructions stored which, when executed by the at least one processor, cause the at least one processor to perform operations comprising: any of the methods described in any preceding clause.

A system comprising: a display; at least one processor; and a non-transitory computer-readable storage medium having instructions stored which, when executed by the at least one processor, cause the at least one processor to perform operations comprising: receiving a plurality of pre-operation medical images of a patient, the plurality of pre-operation medical images capturing at least one vertebral body; segmenting the pre-operation medical images, resulting in three-dimensional (3D) pre-operation model of the at least one vertebral body, the 3D pre-operation model comprising a plurality of volumetric regions, wherein each volumetric region in the plurality of volumetric regions has a bone density based at least in part on the plurality of pre-operation medical images; identifying a plurality of spine surgery options; generating, for each of the plurality of spine surgery options using the 3D pre-operation model, at least one 3D predicted model of the at least one vertebral body, resulting in a plurality of 3D predicted models; simulating a plurality of forces being applied to each model in the plurality of 3D predicted models, resulting in a plurality of predicted surgery outcomes; ranking the plurality of spine surgery options based on the plurality of predicted surgery outcomes, resulting in a plurality of risk assessments; and displaying, via the display, the plurality of risk assessments.

The system of any preceding clause, wherein the generating, for each of the plurality of spine surgery options, of the at least one 3D predicted model further comprises: identifying a plurality of possible locations within the 3D pre-operation model where one or more virtual medical devices can be virtually inserted; and generating, for each of the plurality of possible locations, a distinct model in the plurality of 3D predicted models, such that for each spine surgery option in the plurality of spine surgery options, multiple 3D predicted models are generated, each of the multiple 3D predicted models having the one or more virtual medical devices inserted at distinct locations.

The system of any preceding clause, wherein the distinct locations have distinct bone density.

The system of any preceding clause, wherein the plurality of 3D predicted models further comprise anthropometric data about the patient.

The system of any preceding clause, the non-transitory computer-readable storage medium having additional instructions stored which, when executed by the at least one processor, cause the at least one processor to perform operations comprising: generating, using the plurality of pre-operation medical images and the 3D pre-operation model, a virtual patient model, the virtual patient model comprising virtual representations of the at least one vertebral body and at least one of a ligament, a disc, and a muscle; and generating, for each of the plurality of spine surgery options using the a plurality of 3D predicted models and the virtual patient model, at least one predicted virtual model of the patient, resulting in a plurality of predicted virtual models, wherein the simulating of the plurality of forces is further applied to the plurality of predicted virtual models.

The system of any preceding clause, wherein the simulating is performed by the at least one processor executing a neural network, wherein the neural network is trained on previous spine surgery predictions and associated outcomes.

The system of any preceding clause, the non-transitory computer-readable storage medium having additional instructions stored which, when executed by the at least one processor, cause the at least one processor to perform operations comprising: receiving, after execution of a selected spine surgery option, a plurality of post-operation medical images, the plurality of post-operation medical images capturing the at least one vertebral body; receiving, after execution of the selected spine surgery option, a patient outcome of the selected spine surgery option; segmenting the post-operation medical images, resulting in a 3D post-operation model of the at least one vertebral body; comparing the 3D post-operation model to a specific 3D predicted model within the plurality of 3D predicted models, the specific 3D predicted model being for the selected spine surgery option, resulting in a model comparison; comparing the patient outcome to a predicted surgery outcome within the plurality of predicted surgery outcomes, resulting in an outcome comparison; and updating the neural network based on the model comparison and the outcome comparison.

The system of any preceding clause, wherein the simulating identifies at least one of: (A) a likelihood of vertebrae fracture, (B) a likelihood of subsidence, or (C) a screw pullout risk analysis.

The system of any preceding clause, wherein the bone density for each volumetric region is received in Hounsfield units (HU), then converted by the at least one processor to grams/cubic centimeter (g/cc) of hydroxyapatite.

A non-transitory computer-readable storage medium having instructions stored which, when executed by at least one processor, cause the at least one processor to perform operations comprising: any of the methods described in any preceding clause.

A non-transitory computer-readable storage medium having instructions stored which, when executed by at least one processor, cause the at least one processor to perform operations comprising: receiving a plurality of pre-operation medical images of a patient, the plurality of pre-operation medical images capturing at least one vertebral body; segmenting the pre-operation medical images, resulting in three-dimensional (3D) pre-operation model of the at least one vertebral body, the 3D pre-operation model comprising a plurality of volumetric regions, wherein each volumetric region in the plurality of volumetric regions has a bone density based at least in part on the plurality of pre-operation medical images; identifying a plurality of spine surgery options; generating, for each of the plurality of spine surgery options using the 3D pre-operation model, at least one 3D predicted model of the at least one vertebral body, resulting in a plurality of 3D predicted models; simulating a plurality of forces being applied to each model in the plurality of 3D predicted models, resulting in a plurality of predicted surgery outcomes; ranking the plurality of spine surgery options based on the plurality of predicted surgery outcomes, resulting in a plurality of risk assessments; and displaying, via the display, the plurality of risk assessments.

We claim:

1. A method comprising:
receiving, at a computer system, a plurality of pre-operation medical images of a patient, the plurality of pre-operation medical images capturing at least one vertebral body;
segmenting, via at least one processor of the computer system, the pre-operation medical images, resulting in three-dimensional (3D) pre-operation model of the at least one vertebral body, the 3D pre-operation model comprising a plurality of volumetric regions,
wherein each volumetric region in the plurality of volumetric regions has a bone density based at least in part on the plurality of pre-operation medical images;
identifying a plurality of spine surgery options;
generating, for each of the plurality of spine surgery options using the 3D pre-operation model, at least one 3D predicted model of the at least one vertebral body, resulting in a plurality of 3D predicted models;
simulating, via the at least one processor, a plurality of forces being applied to each model in the plurality of 3D predicted models, resulting in a plurality of predicted surgery outcomes;
ranking, via the at least one processor, the plurality of spine surgery options based on the plurality of predicted surgery outcomes, resulting in a plurality of risk assessments; and
displaying, via a display of the computer system, the plurality of risk assessments.

2. The method of claim 1, wherein the generating, for each of the plurality of spine surgery options, of the at least one 3D predicted model further comprises:
identifying a plurality of possible locations within the 3D pre-operation model where one or more virtual medical devices can be virtually inserted; and
generating, for each of the plurality of possible locations, a distinct model in the plurality of 3D predicted models,
such that for each spine surgery option in the plurality of spine surgery options, multiple 3D predicted models are generated, each of the multiple 3D predicted models having the one or more virtual medical devices inserted at distinct locations.

3. The method of claim 2, wherein the distinct locations have distinct bone density.

4. The method of claim 1, wherein the plurality of 3D predicted models further comprise anthropometric data about the patient.

5. The method of claim 1, further comprising:
generating, via the at least one processor using the plurality of pre-operation medical images and the 3D pre-operation model, a virtual patient model, the virtual patient model comprising virtual representations of the at least one vertebral body and at least one of a ligament, a disc, and a muscle; and
generating, for each of the plurality of spine surgery options using the plurality of 3D predicted models and the virtual patient model, at least one predicted virtual model of the patient, resulting in a plurality of predicted virtual models,
wherein the simulating of the plurality of forces is further applied to the plurality of predicted virtual models.

6. The method of claim 1, wherein the simulating is performed by the at least one processor executing a neural network, wherein the neural network is trained on previous spine surgery predictions and associated outcomes.

7. The method of claim 6, further comprising:
receiving, at the computer system after execution of a selected spine surgery option, a plurality of post-operation medical images, the plurality of post-operation medical images capturing the at least one vertebral body;
receiving, at the computer system after execution of the selected spine surgery option, a patient outcome of the selected spine surgery option;
segmenting, via the at least one processor, the post-operation medical images, resulting in a 3D post-operation model of the at least one vertebral body;
comparing, via the at least one processor, the 3D post-operation model to a specific 3D predicted model within the plurality of 3D predicted models, the specific 3D predicted model being for the selected spine surgery option, resulting in a model comparison;
comparing, via the at least one processor, the patient outcome to a predicted surgery outcome within the plurality of predicted surgery outcomes, resulting in an outcome comparison; and
updating the neural network based on the model comparison and the outcome comparison.

8. The method of claim 1, wherein the simulating identifies at least one of: (A) a likelihood of vertebrae fracture, (B) a likelihood of subsidence, or (C) a screw pullout risk analysis.

9. The method of claim 1, wherein the bone density for each 3D region is received in Hounsfield units (HU), then converted by the at least one processor to grams/cubic centimeter (g/cc) of hydroxyapatite.

10. The method of claim 1, wherein:
the pre-operation medical images further capture at least one functional spinal unit; and
the plurality of 3D predicted models are further based on the at least one functional spinal unit.

11. A system comprising:
a display;
at least one processor; and
a non-transitory computer-readable storage medium having instructions stored which, when executed by the at least one processor, cause the at least one processor to perform operations comprising:
receiving a plurality of pre-operation medical images of a patient, the plurality of pre-operation medical images capturing at least one vertebral body;
segmenting the pre-operation medical images, resulting in three-dimensional (3D) pre-operation model of the at least one vertebral body, the 3D pre-operation model comprising a plurality of volumetric regions,
wherein each volumetric region in the plurality of volumetric regions has a bone density based at least in part on the plurality of pre-operation medical images;
identifying a plurality of spine surgery options;
generating, for each of the plurality of spine surgery options using the 3D pre-operation model, at least one 3D predicted model of the at least one vertebral body, resulting in a plurality of 3D predicted models;
simulating a plurality of forces being applied to each model in the plurality of 3D predicted models, resulting in a plurality of predicted surgery outcomes;
ranking the plurality of spine surgery options based on the plurality of predicted surgery outcomes, resulting in a plurality of risk assessments; and
displaying, via the display, the plurality of risk assessments.

12. The system of claim 11, wherein the generating, for each of the plurality of spine surgery options, of the at least one 3D predicted model further comprises:
identifying a plurality of possible locations within the 3D pre-operation model where one or more virtual medical devices can be virtually inserted; and
generating, for each of the plurality of possible locations, a distinct model in the plurality of 3D predicted models,
such that for each spine surgery option in the plurality of spine surgery options, multiple 3D predicted models are generated, each of the multiple 3D predicted models having the one or more virtual medical devices inserted at distinct locations.

13. The system of claim 12, wherein the distinct locations have distinct bone density.

14. The system of claim 11, wherein the plurality of 3D predicted models further comprise anthropometric data about the patient.

15. The system of claim 11, the non-transitory computer-readable storage medium having additional instructions stored which, when executed by the at least one processor, cause the at least one processor to perform operations comprising:
generating, using the plurality of pre-operation medical images and the 3D pre-operation model, a virtual patient model, the virtual patient model comprising virtual representations of the at least one vertebral body and at least one of a ligament, a disc, and a muscle; and
generating, for each of the plurality of spine surgery options using the a plurality of 3D predicted models and the virtual patient model, at least one predicted virtual model of the patient, resulting in a plurality of predicted virtual models,
wherein the simulating of the plurality of forces is further applied to the plurality of predicted virtual models.

16. The system of claim 11, wherein the simulating is performed by the at least one processor executing a neural network, wherein the neural network is trained on previous spine surgery predictions and associated outcomes.

17. The system of claim 16, the non-transitory computer-readable storage medium having additional instructions stored which, when executed by the at least one processor, cause the at least one processor to perform operations comprising:
receiving, after execution of a selected spine surgery option, a plurality of post-operation medical images, the plurality of post-operation medical images capturing the at least one vertebral body;
receiving, after execution of the selected spine surgery option, a patient outcome of the selected spine surgery option;
segmenting the post-operation medical images, resulting in a 3D post-operation model of the at least one vertebral body;
comparing the 3D post-operation model to a specific 3D predicted model within the plurality of 3D predicted models, the specific 3D predicted model being for the selected spine surgery option, resulting in a model comparison;
comparing the patient outcome to a predicted surgery outcome within the plurality of predicted surgery outcomes, resulting in an outcome comparison; and
updating the neural network based on the model comparison and the outcome comparison.

18. The system of claim 11, wherein the simulating identifies at least one of: (A) a likelihood of vertebrae fracture, (B) a likelihood of subsidence, or (C) a screw pullout risk analysis.

19. The system of claim 11, wherein the bone density for each volumetric region is received in Hounsfield units (HU), then converted by the at least one processor to grams/cubic centimeter (g/cc) of hydroxyapatite.

20. A non-transitory computer-readable storage medium having instructions stored which, when executed by at least one processor, cause the at least one processor to perform operations comprising:
receiving a plurality of pre-operation medical images of a patient, the plurality of pre-operation medical images capturing at least one vertebral body;
segmenting the pre-operation medical images, resulting in three-dimensional (3D) pre-operation model of the at least one vertebral body, the 3D pre-operation model comprising a plurality of volumetric regions,
wherein each volumetric region in the plurality of volumetric regions has a bone density based at least in part on the plurality of pre-operation medical images;
identifying a plurality of spine surgery options;
generating, for each of the plurality of spine surgery options using the 3D pre-operation model, at least one 3D predicted model of the at least one vertebral body, resulting in a plurality of 3D predicted models;
simulating a plurality of forces being applied to each model in the plurality of 3D predicted models, resulting in a plurality of predicted surgery outcomes;
ranking the plurality of spine surgery options based on the plurality of predicted surgery outcomes, resulting in a plurality of risk assessments; and
displaying, via the display, the plurality of risk assessments.
